(19) United States
(12) Patent Application Publication
(10) Pub. No.: US 2010/0056952 A1
(43) Pub. Date: Mar. 4, 2010

(54) In Situ Pressure Monitor and Associated Methods

(76) Inventor: Feng Liu, Salt Lake City, UT (US)

Correspondence Address: THORPE NORTH & WESTERN, LLP., P.O. Box 1219, SANDY, UT, 84091-1219, US

(21) Appl. No.: 12/594,859

(22) PCT Filed: Feb. 29, 2008
(86) PCT No.: PCT/US08/55525

§ 371 Date: Oct. 6, 2009

Related U.S. Application Data
(60) Provisional application No. 60/922,108, filed on Apr. 6, 2007.

(51) Int. Cl. A61B 5/00

(52) U.S. Cl. 600/561

(57) ABSTRACT

Methods, systems, and devices for detecting and quantifying pressure and pressure changes within a system are provided. In one aspect, a pressure sensing device is provided that includes a sensing tube having an interior volume and at least one wall, the wall being configured to deform in response to an external pressure that is greater than an external pressure threshold. The at least one wall is further configured to deform as a function of the external pressure. The device may also include a transducer operably coupled to the sensing tube, the transducer being configured to detect changes in the interior volume as a result of deformation of the at least one wall.

### FIELD OF THE INVENTION

The present invention relates generally to pressure sensing devices and associated methods. Accordingly, the present invention involves the material science, medicine, mechanical engineering, and physics fields.

### BACKGROUND OF THE INVENTION

In many biological systems internal pressure can be an indicator of a variety of medical conditions that may require monitoring to provide appropriate medical treatment. In many cases it would be beneficial to continuously monitor pressure in a system in order to quickly make adjustments to the medical treatment as internal pressure changes. The ability to perform such continuous monitoring may be hampered by large, bulky pressure sensing devices. Additionally, many of the current pressure sensing devices available were originally designed for sensing relatively high pressures, and may not be highly accurate at the pressure levels observed in many biological systems.

Many current pressure sensing devices are based on conventional mechanisms for sensing pressure. One example of such a mechanism includes measuring the bending of a micrometer thin solid film under pressure. Such a micro-electromechanical system (MEMS) functions by measuring a capacitance change in a built-in inductor-capacitor circuit as a result of bending or displacement of the solid film. In addition to being bulky and complex, these devices may prove unsuitable for implantation and long-term retention in a subject.

### SUMMARY OF THE INVENTION

The present invention provides methods, systems, and devices for detecting and quantifying pressure and pressure changes within a system. In one aspect, for example, a pressure sensing device is provided. Such a device may include a sensing tube having an interior volume and at least one wall, where the wall is configured to deform in response to an external pressure that is greater than an external pressure threshold, and wherein the at least one wall is configured to deform as a function of the external pressure. The device may further include a transducer operably coupled to the sensing tube, where the transducer is configured to detect changes in the interior volume as a result of deformation of the at least one wall.

It is contemplated that the tube may be of any size that is beneficial for the detection of pressure in a system, and that tube size may vary depending on the intended use of the device. In one aspect, however, the tube is a microtube having a cross-sectional diameter of from about 1 micron to about 2000 microns. In another aspect, the tube is a microtube having a cross-sectional diameter of from about 20 microns to about 200 microns. In yet another aspect, the tube is a microtube having a cross-sectional diameter of from about 100 microns to about 1000 microns. In a further aspect, the tube is a microtube having a cross-sectional diameter of from about 1000 microns to about 2000 microns.

Additionally, the tube may be constructed of a variety of materials, and as such, the materials described should not be seen as limiting. In one aspect, however, the sensing tube is a polymeric tube. Non-limiting examples of suitable polymeric materials may include polyethylenes, polyurethanes, polyurethane elastomers, silicone-hydrogels, polyimides, polyetheretherketones, polytetrafluoroethylenes, polyethylenes, polydimethylsiloxanes, etc.

The present invention additionally provides a system for sensing pressure, including a pressure sensing device having a sensing tube with an interior volume and at least one wall, where the wall is configured to deform in response to an external pressure that is greater than an external pressure threshold, and where the at least one wall is configured to deform as a function of the external pressure. The pressure sensing device may further include a transducer operably coupled to the sensing tube, where the transducer is configured to detect changes in the interior volume as a result of deformation of the at least one wall. Additionally, the system may include a data acquisition system operably coupled to the transducer where the data acquisition device is configured to receive a pressure monitor signal from the transducer.

The present invention further provides a method for sensing pressure within a system, including delivering a pressure sensing device into the system, where the pressure sensing device further includes a sensing tube having an interior volume and at least one wall, where the wall is configured to deform in response to an external pressure that is greater than an external pressure threshold, and where the at least one wall is configured to deform as a function of the external pressure. The pressure sensing device may further include a transducer operably coupled to the sensing tube, where the transducer is configured to detect changes in the interior volume as a result of deformation of the at least one wall. The method may also include detecting a change in the interior volume as a result of a change in the external pressure that is greater than the external pressure threshold. In another aspect, the method may also include quantifying a degree of the change in the external pressure by detecting a degree of the change in the interior volume. In yet another aspect, the method may include transmitting the change in the interior volume to a data acquisition system.

There has thus been outlined, rather broadly, various features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### BRIEF DESCRIPTION OF THE DRAWINGS

FIG. 1 is a graphical representation of the effects of pressure on a tube in accordance with one exemplary embodiment of the present invention;

FIG. 2 is a graphical representation of the effects of pressure on a tube in accordance with an exemplary embodiment of the present invention; and

FIG. 3 is a cross-section view of an exemplary pressure sensing device in accordance with an embodiment of the present invention.

### DETAILED DESCRIPTION OF THE INVENTION

### Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a microtube” includes reference to one or more of such microtubes, and reference to “the transducer” includes reference to one or more of such transducers.

As used herein, the term “interior volume” refers to a volume on an inside of a pressure sensing tube. The interior volume may be a measurement of all of the volume contained within the tube, or it may be a measurement of only a portion of the volume contained within the tube. For example, one method of measuring interior volume change may be accomplished by partially filling a tube with a liquid, and measuring changes in the level of the liquid within the tube as external pressure changes. It is recognized that references to changes in interior volume may not actually be volumetric changes in a closed tube, but rather may be a detectable displacement of a liquid or other medium within the tube, be it closed or open.

As used herein, the term “external pressure” refers to the pressure exerted on the exterior of the tube. As such, a pressure sensing tube implanted within an organ system would experience external pressure from the internal pressure of the organ system.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

The present invention provides devices, systems, and methods for detecting pressure in a variety of systems. Although such systems may include any type of system known, these teachings are particularly valuable in biological systems due to the small changes in pressure that are often indicative of various medical conditions that may require monitoring or treatment. It has now been discovered that various tube structures may be utilized to detect such changes in pressure, where the tubes can be of a size that is small enough to avoid detrimental or irritating side effects associated with implantation. In one aspect, for example, such a pressure sensing device can include a sensing tube having an interior volume and at least one wall, where the wall is configured to deform in response to an external pressure that is greater than an external pressure threshold. Additionally, the at least one wall can be configured to deform as a function of the external pressure. The device can further include a transducer operably coupled to the sensing tube, where the transducer is configured to detect changes in the interior volume as a result of deformation of the at least one wall.

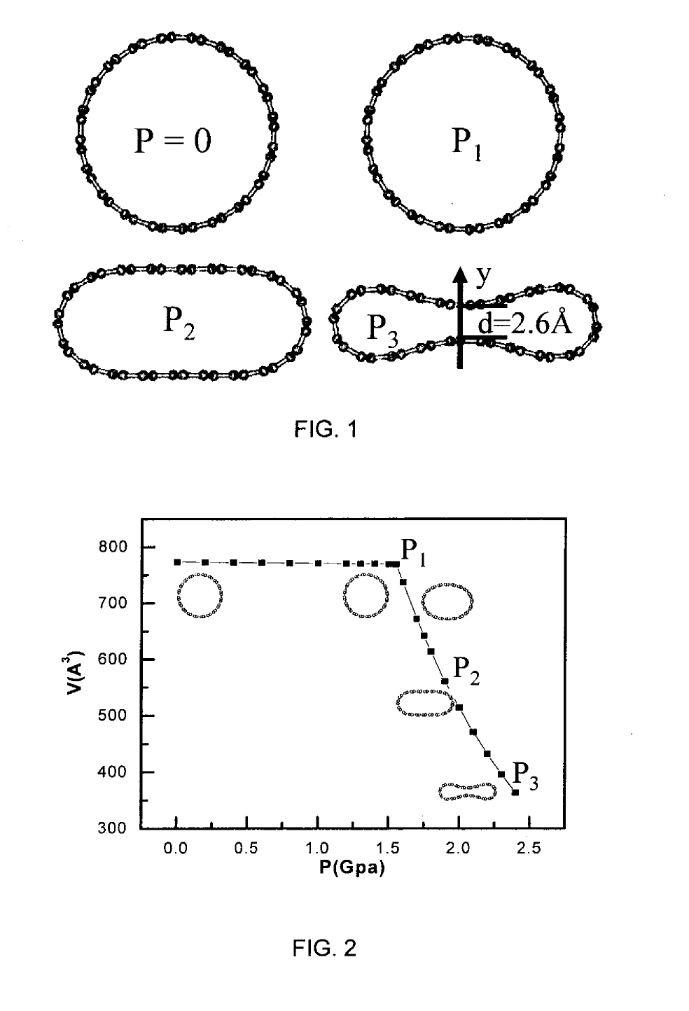

The tubes and microtubes according to aspects of the present invention exhibit structural deformations under pressure that can be beneficially utilized as pressure sensors in a variety of biological as well as non-biological systems. Such tubes undergo a series of shape transitions as external pressure is increases. FIG. 1 shows a molecular dynamic simulation of the structural deformation of a carbon nanotube as external pressure in increased, where P₁=1.6 GPa, P₂=1.5 GPa, and P₃=2.4 GPa. These deformations are structural manifestations of volume changes occurring inside the tubes as a result of the changes in external pressure. By quantifying the degree of volume change within the tube, an accurate estimation of the pressure external to the tube can thus be obtained. Interestingly, it has been discovered that the structural deformation properties are very similar across tubes of different sizes and constructions, from carbon nanotubes to polymeric macrotubes.

Furthermore, it has been discovered that two distinct transition regimes occur as external pressure increases that can be utilized to facilitate the detection and quantification of changes in pressure. These two transition regimes are uniquely defined by the tube geometric dimensions and physical properties such as elastic constants, and as such, pressure sensors can be designed and fabricated to detect and quantify pressures within specific desired ranges. The first transition regime is the transition pressure threshold. As can be seen in FIG. 2, the volume and thus the wall structure of a tube is maintained as pressure is increased up to a transition pressure threshold or critical transition pressure (P_{c}), as defined in Equation I:

$${P}_{c} = \frac{3 D}{{R}^{3}} \qquad I$$

where R is the radius of the tube at zero pressure, and D is the flexural rigidity of the tube, a constant related to the modulus and Poisson ratio of the tube. Thus, the larger the radius of the tube, the lower the P_{c}. At P_{c} the tube begins to exhibit measurable volume change due to the deformation of the tube wall, as it is easier to bend than to compress the tube. This leads to shape instability, transforming the tube from an isotropic circular shape to an anisotropic elliptical shape. At pressures below P_{c} the tube is in a “hard phase” and the tube volume remains effectively constant. Above P_{c} the tube is in a “soft phase” and the tube volume decreases relative to increasing pressure. This hard-to-soft phase transition provides a mechanism to define a threshold pressure for monitoring a selected range of pressures. P_{c} can thus be adjusted through the design and fabrication of the device to facilitate activation of the sensor at a desired external pressure. For example, P_{c} can be adjusted by altering the radius and/or the wall thickness of the tube.

Such a tube sensor can be useful in a variety of biological and non-biological systems. For example, in many organ systems in a subject, pressure may normally fluctuate within a normal or acceptable range. A pressure sensor tube can therefore be designed and fabricated such that the threshold for P_{c} is above the acceptable pressure fluctuation range, and thus the sensor will not activate until pressure within the organ system has increased beyond the established threshold. As one specific example, a tube can be designed having P_{c} set at approximately 2800 Pa, corresponding to the lower limit of the disease state of glaucoma. This tube sensor embedded in the eye would thus not activate and begin sensing pressure until the intraocular pressure reached at least 2800 Pa.

The second transition regime is a pressure-volume relationship that occurs at pressures greater than the transition pressure threshold. As can be seen in FIG. 2, the volume inside the pressure sensing tube decreases as function of increasing pressure external to the tube. Thus once the external pressure has increased to the level defined by P_{c}, further pressure increases can be quantified according to the decrease in tube volume to determine the degree of pressure increase.

The size and configuration of tubes according to aspects of the present invention can vary depending on the intended use and functioning location of the pressure sensing device. In one aspect, however, the tube can be a microtube. In a more specific aspect, the tube can have a cross-sectional diameter of from about 1 micron to about 2000 microns. In another aspect, the tube can have a cross-sectional diameter of from about 20 microns to about 200 microns. In yet another aspect, the tube can have a cross-sectional diameter of from about 100 microns to about 1000 microns. In a further aspect, the tube can have a cross-sectional diameter of from about 1000 microns to about 2000 microns. Additionally, the tubes can be made to a variety of lengths, depending on the intended use of the sensor.

In addition to tube size and length, various materials are contemplated that can be used to construct pressure sensing tubes. It should be noted that the scope of the claims of the present invention should not be limited by the materials used to construct the tube, and that the materials described herein are intended to be exemplary. That being said, polymeric materials can be utilized to construct sensing tubes of any size that deform under pressure according to the aforementioned description. Polymeric tubes of an appropriate size and shape can be purchased readily. In those instances where tubes of a particular polymeric material are unavailable commercially, well known methods are available to allow one of ordinary skill in the art to make such devices. Accordingly, in one aspect, the polymeric tube can be made from polymeric materials such as polyethylenes, polyurethanes, polyurethane elastomers, silicone-hydrogels, polyimides, polyetheretherketones, polytetrafluoroethylenes, polyethylenes, polydimethylsiloxanes, etc.

Furthermore, in one aspect the pressure sensing device can include a plurality of sensing tubes, where each sensing tube has a distinct or different pressure threshold. Such a configuration can be beneficial for very broad pressure detection ranges or for pressure detection regimes where multiple thresholds and ranges require monitoring.

The pressure sensing devices of the present invention additionally include a transducer to transduce the pressure induced volume change within the tube into a signal that can be transmitted remote from the sensor. The transducer can be made in a variety of sizes, provided the size does not interfere with the functioning of the device. It can be beneficial, however, to utilize small transducers that are very sensitive to volume change because the transducer is often coupled to the pressure sensing tube that is implanted in a biological system. The smaller the size of the device, the less the detrimental impact will be on the biological system receiving the device.

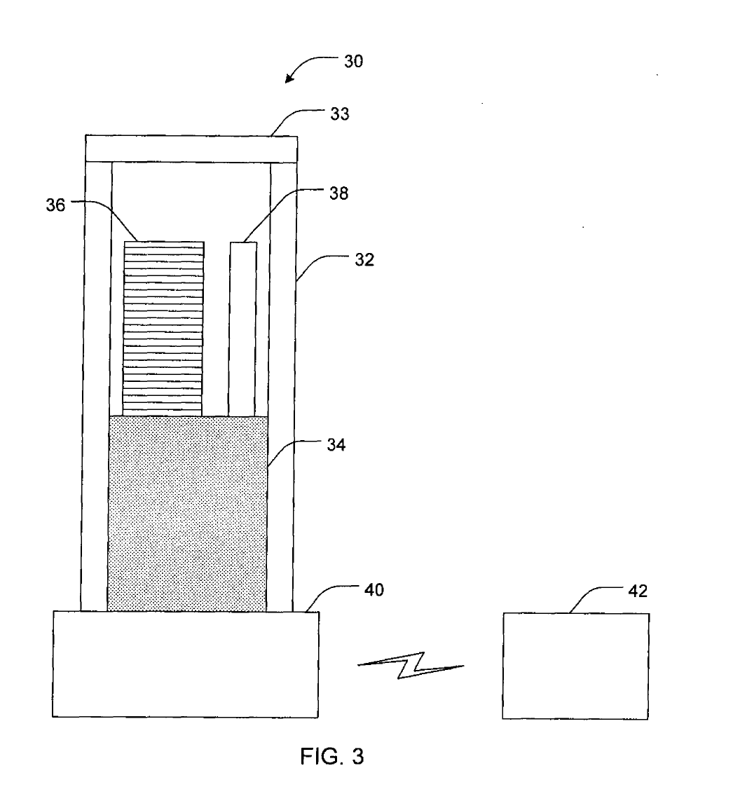

Any transduction method that can be utilized in conjunction with the pressure sensing tubes of the present invention should be considered to be within the present scope. Non-limiting examples of such transduction methods can include piezoelectric, piezoresistive, resistive, capacitative, optical, reflectometerical, etc. A number of transducers are commercially available that could be used. In one specific aspect, however, a microlevel liquid sensor can be used to transduce the volume change within the tube. In such cases, a microwire, a thin film resistor, or an interdigitated electrode structure (IDE) can be used to measure liquid level by tracking changes in capacitance, by using reflectometry, or by measuring changes in resistance due to changes in liquid level. One example of such a sensor is shown in FIG. 3. Such a sensor 30 can include a tube 32 configured to deform under pressure as described herein. The tube 32 is shown with a tube cap 33 that effectively sealing the tube from the external environment, however the tube can be sealed by other methods such as crimping, twisting, etc. The tube 32 can be filled with a liquid 34 to provide a measurement of volume change. Non-limiting examples of appropriate liquids can include physiological solutions such as 0.9% NaCl, or various buffers such as PBS buffer. A resistive level sensor including a micromachined IDE contact structure 36 and a microwire 38 is positioned in the tube 32 to measure the level of the liquid 34. When the pressure outside of the tube 32 increases, volume changes will cause the level of the liquid 34 to rise, and such changes can be detected by the IDE contact structure 36. A measurement system 40 coupled to the IDE contact structure 36 measures changes in the properties of the IDE contact structure 36 and transmits such measurements to a location remote from the tube sensor. Such a remote location can be a data acquisition system 42 designed to acquire pressure measurements from the sensing device. Transmission may be by one or more of a variety of means known, such as wire, wireless, etc. The resolution of the sensor depends on the resolution of the IDE transducer in the case of resistive sensors, or in the frequency and conductivity of the liquid in the case of wire/reflectometry sensors. The total resistance of the wire resistor decreases since the liquid shorts out the submerged portion of the wire. In order to improve the sensitivity the wire resistor to the liquid level, the resistance of the bottom portion of the wire should be maximized. The accuracy of the level measurement can depend on the minimum feature size of the WDE or wire structure.

It can also be beneficial for the pressure sensing device to be biologically inert. This can be accomplished by utilizing biologically inert materials to construct the device, or it can be accomplished by coating exposed surfaces of the pressure sensing device with a layer of a biologically inert material. The type of biologically inert material used can vary widely depending on the configuration of the sensor device and the intended duration of use in the biological system. Biologically inert materials are well known in the art, and the use of such materials is well within the knowledge of one of ordinary skill in the art.

The present invention additionally provides systems for sensing pressure. In one example aspect, such a system can include a pressure sensing device having a sensing tube with an interior volume and at least one wall, the wall being configured to deform in response to an external pressure that is greater than an external pressure threshold. Additionally, the at least one wall is configured to deform as a function of the external pressure. The system can further include a transducer operably coupled to the sensing tube, where the transducer is configured to detect changes in the interior volume as a result of deformation of the at least one wall. Furthermore, the system also includes a data acquisition system operably coupled to the transducer, where the data acquisition device is configured to receive a pressure monitor signal from the transducer. As has been described, the data acquisition system can be operatively coupled to the transducer by a variety of mechanisms, including physical coupling such as wired coupling, and non-physical coupling such as wireless or optical coupling.

The present invention additionally provides methods for sensing pressure within a system. Such a method can include delivering a pressure sensing device into the system, where the pressure sensing device further includes a sensing tube having an interior volume and at least one wall, where the wall is configured to deform in response to an external pressure that is greater than an external pressure threshold, and the at least one wall is further configured to deform as a function of the external pressure. The pressure sensor can additionally include a transducer operably coupled to the sensing tube, where the transducer is configured to detect changes in the interior volume as a result of deformation of the at least one wall. Following delivery of the pressure sensing device into the system, the method can further include detecting a change in the interior volume of the tube as a result of a change in the external pressure that is greater than the external pressure threshold. In another aspect, the method can include quantifying a degree of the change in the external pressure by detecting a degree of the change in the interior volume. Such quantification can be derived as described herein through the pressure-to-volume ratio changes that occur in response to external pressure. Furthermore, in another aspect the method can include transmitting the change in the interior volume to a data acquisition system.

As has been described, the pressure sensing devices according to aspects of the present invention can be utilized to detect and quantify pressure in a variety of biological and non-biological systems. It should be noted that the scope of the present invention should not be limited to the specific systems described herein. Additionally, the configuration and design of the sensing device can vary depending on the system into which such a device is introduced.

In one aspect of the present invention, for example, a tubular pressure sensing device can be utilized to detect and quantify increases in ocular pressure as a result of an ocular condition such as glaucoma. Glaucoma is an ocular disease that is characterized by damage to the optic nerve typically caused by elevated intraocular pressure (IOP). Although there is not a known cure for glaucoma, it is estimated that about 90% of sight loss can be prevented by early detection and treatment to control the level of IOP in the eye. Treatments tend to involve medication, laser therapy, and surgery. Many treatments, particularly those of a surgical nature, are difficult to manage due to the lack of in situ continuous measurement techniques for IOP. For example, many surgical treatments involve draining fluid from the eye in order to reduce IOP. It can be difficult, however, to regulate a correct drainage amount from the eye without an accurate estimation of IOP. Even treatments utilizing medication treatments can prove difficult because the optimal dosage and timing of drugs may not be accurate without knowing IOP.

Accordingly, a pressure sensing device according to aspects of the present invention can be inserted into the eye to allow continuous pressure monitoring for glaucoma treatment. The device can be inserted into the eye by any means known, including by surgical implantation, injection, etc. The small size of the pressure sensing device can allow the device to be maintained in the eye with minimal adverse effects on vision, discomfort, or ocular damage. Following insertion into the eye, the pressure sensing tube can monitor the pressure within the eye and transmit IOP data to a remote recording or acquisition device. In one aspect of the present invention, wireless transmission of IOP data from the eye to the remote acquisition device can be implemented, particularly for those aspects where continuous monitoring is desired. It is contemplated, however, that a wired tether can be utilized in some aspects where the placement of the device is intended to be temporary as in, for example, a surgical procedure.

In another aspect of the present invention, a tubular pressure sensing device can be utilized to detect and quantify increases in intra-abdominal pressure. Abnormal intra-abdominal pressure (IAP) increases may occur in individuals with acute abdominal syndromes such as ileus, intestinal perforation, peritonitis, acute pancreatitis, or trauma. Normal IAP levels are generally from 0-5 mmHg in humans. Elevated IAP may lead to intra-abdominal hypertension (IAH) and abdominal compartment syndrome (ACS), both of which may be related to an increased morbidity and mortality of critically ill individuals. For comparison, intra-abdominal hypertension can include IAP levels that are greater than 12 mmHg. It is also believed that increases in IAP may be associated with various additional forms of organ dysfunction. An increase in IAP may also lead to distal effects in other parts of the body, such as increased intracranial pressure, pericardial tamponade, tension pneumothorax, extremity compartment syndrome, etc. Intra-abdominal placement of a pressure sensor according to aspects of the present invention can thus allow continuous monitoring of abdominal pressure in susceptible individuals, subsequently facilitating the treatment and prevention of various disorders associated with IAP.

As another example, the monitoring of intracranial pressure is important in the management of head trauma and many neural disorders. Edema associated with many pathologic conditions of the brain may cause an increase in intracranial pressure that may in turn lead to secondary neurological damage. In addition to head trauma, various neurological disorders may also lead to increased intracranial pressure. Examples of such disorders can include intracerebral hematoma, subarachnoid hemorrhage, hydrocephalic disorders, infections of the central nervous system, and various lesions to name a few.

As a specific example, hydrocephalus is characterized by increased intracranial pressure due to an excess of cerebrospinal fluid, which is often the result of malabsorpition or impediment of clearance in the intraventricular space within the brain or subarachnoid spaces about the brain. Hydrocephalus is often treated by insertion of a diverting catheter into the ventricles of the brain or into the lumbar cistern. Such a catheter or shunt is connected by a regulating valve to a distal catheter which shunts the spinal fluid to another space where it can be reabsorbed. Measurements of intracranial pressure are critical to the treatment and subsequent monitoring of hydrocephalus and other neurological conditions associated with pressure increases. Such measurements can be accomplished by inserting a pressure sensing tube device into an intraventricular space within the brain to allow direct monitoring of intracranial pressure.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A pressure sensing device, comprising:
a sensing tube having an interior volume and at least one wall, the wall being configured to deform in response to an external pressure that is greater than an external pressure threshold, such that the at least one wall is configured to deform as a function of the external pressure; and
a transducer operably coupled to the sensing tube, the transducer being configured to detect changes in the interior volume as a result of deformation of the at least one wall.

2. The device of claim 1, wherein the tube is a microtube having a cross-sectional diameter of from about 1 micron to about 2000 microns.

3. The device of claim 1, wherein the tube is a microtube having a cross-sectional diameter of from about 20 microns to about 200 microns.

4. The device of claim 1, wherein the tube is a microtube having a cross-sectional diameter of from about 100 microns to about 1000 microns.

5. The device of claim 1, wherein the tube is a microtube having a cross-sectional diameter of from about 1000 microns to about 2000 microns.

6. The device of claim 1, wherein the sensing tube is a polymeric tube.

7. The device of claim 6, wherein the polymeric tube includes a material selected from the group consisting of polyethylenes, polyurethanes, polyurethane elastomers, silicone-hydrogels, polyimides, polyetheretherketones, polytetrafluoroethylenes, polyethylenes, polydimethylsiloxanes, and combinations thereof.

8. The device of claim 1, wherein the transducer includes a transduction mechanism selected from the group consisting of piezoelectric, piezoresistive, resistive, capacitative, optical, reflectometerical, and combinations thereof.

9. The device of claim 1, wherein the pressure sensing device is configured to be biologically inert.

10. The device of claim 9, wherein the at least a portion of the pressure sensing device is coated with a layer of a biologically inert material.

11. The device of claim 1, further comprising a plurality of sensing tubes, each having a distinct pressure threshold.

12. A system for sensing pressure, comprising:
a pressure sensing device including:
a sensing tube having an interior volume and at least one wall, the wall being configured to deform in response to an external pressure that is greater than an external pressure threshold, such that the at least one wall is configured to deform as a function of the external pressure; and
a transducer operably coupled to the sensing tube, the transducer being configured to detect changes in the interior volume as a result of deformation of the at least one wall;
a data acquisition system operably coupled to the transducer, the data acquisition system being configured to receive a pressure indicative signal from the transducer.

13. The system of claim 12, wherein the data acquisition system is wirelessly coupled to the transducer.

14. The system of claim 12, wherein the data acquisition system is physically coupled to the transducer.

15. A method for sensing pressure within a system, comprising:
delivering a pressure sensing device into the system, the pressure sensing device including:
a sensing tube having an interior volume and at least one wall, the wall being configured to deform in response to an external pressure that is greater than an external pressure threshold, such that the at least one wall is configured to deform as a function of the external pressure;
a transducer operably coupled to the sensing tube, the transducer being configured to detect changes in the interior volume as a result of deformation of the at least one wall; and
detecting a change in the interior volume as a result of a change in the external pressure that is greater than the external pressure threshold.

16. The method of claim 15, further comprising quantifying a degree of the change in the external pressure by detecting a degree of the change in the interior volume.

17. The method of claim 15, wherein detecting the change in the interior volume occurs by a transduction mechanism selected from the group consisting of piezoelectric, piezoresistive, resistive, capacitative, optical, reflectometerical, and combinations thereof.

18. The method of claim 15, further comprising transmitting a signal representative of the change in the interior volume to a data acquisition system.

19. The method of claim 15, wherein the system is a biological system.

20. The method of claim 15, wherein the external pressure is intra-abdominal pressure.
